(19) United States
(12) Patent Application Publication
(10) Pub. No.: US 2025/0123259 A1
(43) Pub. Date: Apr. 17, 2025

(54) SYSTEM AND METHOD FOR ANALYZING GAS COMPOSITION CONTENT OF TRANSFORMER OIL

(71) Applicant: LANSIS INSTRUMENT (SHANGHAI) CO., LTD, Shanghai (CN)

(72) Inventors: Chaoqing LI, Shanghai (CN); Fude WANG, Shanghai (CN); Jianhao LI, Shanghai (CN)

(21) Appl. No.: 18/828,367

(22) Filed: Sep. 9, 2024

(30) Foreign Application Priority Data
Oct. 12, 2023 (CN) 202311321752.3

(51) Int. Cl. G01N 33/28; G01N 30/02; G01N 30/32; G01N 30/66; G01N 30/68; G01N 30/86

(52) U.S. Cl. CPC G01N 33/2841; G01N 30/32; G01N 30/66; G01N 30/68; G01N 30/8631; G01N 2030/025; G01N 2030/328

(57) ABSTRACT

System and method for analyzing gas composition content of transformer oil are provided. The analysis system includes a ten-way switching valve, a sample loop, a first chromatographic column, a second chromatographic column, a thermal conductivity cell detector, a four-way switching valve, a third chromatographic column, and a carbon composition detection device. A first composition fitted peak and a second composition fitted peak are sequentially separated from a transformer oil sample by the first chromatographic column, the first composition fitted peak is a fitted peak of hydrogen, oxygen, and nitrogen; and the second composition fitted peak is a fitted peak of carbon monoxide, methane, carbon dioxide, and a C₂ hydrocarbon compositions.

### CROSS-REFERENCE TO RELATED APPLICATION

This patent application claims the benefit and priority of Chinese Patent Application No. 202311321752.3 filed with the China National Intellectual Property Administration on Oct. 12, 2023, the disclosure of which is incorporated by reference herein in its entirety as part of the present application.

### TECHNICAL FIELD

The present disclosure relates to the field of gas chromatography determination, and in particular to a system and a method for analyzing gas composition content of transformer oil.

### BACKGROUND

At present, the principle of chromatographic gas flow published in DT/T703-2015 Determination of dissolved gas content in insulating oil by gas chromatography is that manual injection is adopted and one sample needs to be injected twice, leading to complicated operation, or headspace injection needs to cooperate with valve switching mode injection, the sample needs to be subjected to pretreatment and isothermal equilibrium during headspace injection, making the operating conditions demanding.

Therefore, it is necessary to simplify the determination process of gas chromatography under the condition of ensuring accurate determination results.

### SUMMARY

An objective of the present disclosure is to provide a system and a method for analyzing gas composition content of transformer oil, which can achieve the detection of contents of hydrogen, oxygen, nitrogen, carbon monoxide, methane, carbon dioxide, acetylene, ethylene and ethane compositions in the transformer oil by a single injection.

To achieve the objective above, the present disclosure employs the following technical solution:

A system for analyzing gas composition content of transformer oil includes a ten-way switching valve, a sample loop, a first chromatographic column, a second chromatographic column, a thermal conductivity cell detector, a four-way switching valve, a third chromatographic column, and a carbon composition detection device.

A first port of the ten-way switching valve is a transformer oil sample inlet, a tenth port of the ten-way switching valve is connected to one end of the sample loop, and a third port of the ten-way switching valve is connected to an other end of the sample loop.
a fourth port of the ten-way switching valve is a first carrier gas inlet, a ninth port of the ten-way switching valve is connected to one end of the first chromatographic column, and a sixth port of the ten-way switching valve is connected to an other end of the first chromatographic column. A fifth port of the ten-way switching valve is connected to one end of the second chromatographic column, and the thermal conductivity cell detector is connected to an other end of the second chromatographic column. The first chromatographic column is used to separate a dissolved gas in the transformer oil sample to obtain a first composition fitted peak and a second composition fitted peak in sequence. The first composition fitted peak is a fitted peak of hydrogen, oxygen, and nitrogen; the second composition fitted peak is a fitted peak of carbon monoxide, methane, carbon dioxide and a C₂ hydrocarbon compositions. The second chromatographic column is used to separate from the first composition fitted peak to obtain hydrogen, oxygen, and nitrogen. The thermal conductivity cell detector is used to detect the contents of hydrogen, oxygen and nitrogen, respectively.

A seventh port of the ten-way switching valve is a second carrier gas inlet, an eighth port of the ten-way switching valve is connected to a first port of the four-way switching valve, a second port of the four-way switching valve is connected to one end of the third chromatographic column, and an other end of the third chromatographic column is connected to the carbon composition detection device. The third chromatographic column is used to separate from the second composition fitted peak to obtain carbon monoxide, methane, carbon dioxide, ethylene, ethane, and acetylene. The carbon composition detection device is used to detect contents of carbon monoxide, methane, carbon dioxide, ethylene, ethane and acetylene, respectively.

A method for analyzing gas composition content of transformer oil includes:
a sample collection phase:
setting an interface state of a ten-way switching valve to a first interface connection mode, and setting an interface state of a four-way switching valve to a first connection state, where the first interface connection mode is that, for the ten-way switching valve, a first port communicates with a tenth port, a second port communicates with a third port, a fourth port communicates a fifth port, a sixth port communicates with a seventh port, and an eighth port communicates with a ninth port; and the first connection state is that, for the four-way switching valve, a first port communicates with a fourth port, and a second port communicates with a third port;
introducing a transformer oil sample from the first port of the ten-way switching valve, and enabling the transformer oil sample to flow to a sample loop;
a hydrogen, oxygen and nitrogen composition detection phase:
setting the interface state of the ten-way switching valve to a second interface connection mode, where the second interface connection mode is that, for the ten-way switching valve, the first port communicates with the second port, the third port communicates with the fourth port, the fifth port communicates with the sixth port, the seventh port communicates with the eighth port, and the ninth port communicates with the tenth port;
introducing a first carrier gas from the fourth port of the ten-way switching valve, carrying, by the first carrier gas, the transformer oil sample in the sample loop to enter a first chromatographic column, and separating a first composition fitted peak and a second composition fitted peak in sequence from the transformer oil sample, where the first composition fitted peak is a fitted peak of hydrogen, oxygen and nitrogen, and the second composition fitted peak is a fitted peak of carbon monoxide, methane, carbon dioxide, and a C₂ hydrocarbon compositions;
after the first composition fitted peak completely enters a second chromatographic column, switching the four-way switching valve to a second connection state, and after hydrogen, oxygen and nitrogen are obtained by separating the first composition fitted peak by the second chromatographic column, respectively detecting the contents of hydrogen, oxygen and nitrogen by a thermal conductivity cell detector, where the second connection state is that, for the four-way switching valve, the first port communicates with the second port, and the third port communicates with the fourth port; and
a carbon monoxide, methane, carbon dioxide and C₂ hydrocarbon composition detection phase:
switching the interface state of the ten-way switching valve to the first interface connection mode; and
introducing a second carrier gas from the seventh port of the ten-way switching valve, carrying, by a second carrier gas, the second composition fitted peak to completely enter a third chromatographic column, wherein the second composition fitted peak is separated by the third chromatographic column and then enters a carbon composition detection device for determining the contents of carbon monoxide, methane, carbon dioxide and C₂ hydrocarbon.

According to specific embodiments of the present disclosure, the present disclosure has the following technical effects:

According to the system and method for analyzing gas composition content of transformer oil provided by the present disclosure, a first composition fitted peak and a second composition fitted peak are sequentially separated from a transformer oil sample by a first chromatographic column. According to a separation order, the contents of gases in the separated first composition fitted peak are detected by a second chromatographic column and the thermal conductivity cell detector, and then the contents of gases in the separated second composition fitted peak are detected by a third chromatographic column and the carbon composition detection device. Therefore, the contents of hydrogen, oxygen, nitrogen, carbon monoxide, methane, carbon dioxide, acetylene, ethylene and ethane in the transformer oil can be detected by a single injection.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a connection schematic diagram of a system for analyzing gas composition content in transformer oil according to an embodiment of the present disclosure in a sample collection phase;

FIG. 2 is a connection schematic diagram of a system for analyzing gas composition content in transformer oil according to an embodiment of the present disclosure in a hydrogen, oxygen and nitrogen composition detection phase;

FIG. 3 is a connection schematic diagram of a system for analyzing gas composition content in transformer oil according to an embodiment of the present disclosure in a C₃₊ hydrocarbon compositions venting phase.

In the drawings: 1—sample storage tank; 2—first pressure relief valve; 3—sample loop; 4—tail gas recovery unit; 5—first chromatographic column; 6—first carrier gas; 7—second chromatographic column; 8—thermal conductivity cell detector; 9—second carrier gas; 10—ten-way switching valve; 11—first planar tee joint; 12—second planar tee joint; 13—second pressure relief valve; 14—carrier gas storage device; 15—hydrogen flame ionization detector; 16—needle valve; 17—four-way switching valve; 18—third carrier gas; 19—nickel converter device; 20—third chromatographic column.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

In order to make the objectives, features and advantages of the present disclosure more clearly, the present disclosure is further described in detail below in conjunction with the accompanying drawings and specific embodiments.

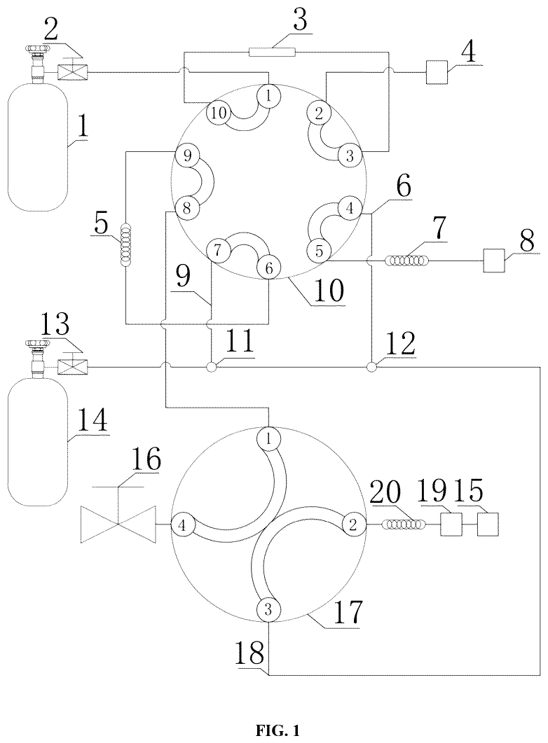

As shown in FIG. 1, this embodiment provides a system for analyzing gas composition content of transformer oil, including a ten-way switching valve 10, a sample loop 3, a first chromatographic column 5, a second chromatographic column 7, a thermal conductivity cell detector 8, a four-way switching valve 17, a third chromatographic column 20, and a carbon composition detection device.

A first port of the ten-way switching valve 10 is a transformer oil sample inlet, a tenth port of the ten-way switching valve 10 is connected to one end of the sample loop 3, and a third port of the ten-way switching valve 10 is connected to the other end of the sample loop 3.

A fourth port of the ten-way switching valve 10 is a first carrier gas 6 inlet, a ninth port of the ten-way switching valve 10 is connected to one end of the first chromatographic column 5, and a sixth port of the ten-way switching valve 10 is connected to the other end of the first chromatographic column 5. A fifth port of the ten-way switching valve 10 is connected to one end of the second chromatographic column 7, and the thermal conductivity cell detector 8 is connected to the other end of the second chromatographic column 7. The first chromatographic column 5 is used to separate a dissolved gas in the transformer oil sample to obtain a first composition fitted peak and a second composition fitted peak in sequence. The first composition fitted peak is a fitted peak of hydrogen, oxygen, and nitrogen. The second composition fitted peak is a fitted peak of carbon monoxide, methane, carbon dioxide and a C₂ hydrocarbon compositions. The second chromatographic column 7 is used to separate from the first composition fitted peak to obtain hydrogen, oxygen, and nitrogen. The thermal conductivity cell detector 8 is used to detect the contents of hydrogen, oxygen and nitrogen, respectively.

A seventh port of the ten-way switching valve 10 is a second carrier gas 9 inlet, an eighth port of the ten-way switching valve 10 is connected to a first port of the four-way switching valve 17, a second port of the four-way switching valve 17 is connected to one end of the third chromatographic column 20, and the other end of the third chromatographic column 20 is connected to the carbon composition detection device. The third chromatographic column 20 is used to separate from the second composition fitted peak to obtain carbon monoxide, methane, carbon dioxide, ethylene, ethane, and acetylene. The carbon composition detection device is used to detect the contents of carbon monoxide, methane, carbon dioxide, ethylene, ethane and acetylene, respectively.

In an example, the carbon composition detection device includes a nickel converter device 19, and a hydrogen flame ionization detector 15. One end of the nickel converter device 19 is connected to the other end of the third chromatographic column 20, and an other end of the nickel converter device 19 is connected to the hydrogen flame ionization detector 15. The nickel converter device 19 is used to hydrogenate carbon monoxide and carbon dioxide under a catalysis of a nickel catalyst to generate methane. The hydrogen flame ionization detector 15 is used to detect the contents of methane, acetylene, ethylene, and ethane, and to detect the contents of carbon monoxide and carbon dioxide in the generated methane.

In another example, the analysis system further includes a needle valve 16. The needle valve 16 is connected to a fourth port of the four-way switching valve 17. After the second composition fitted peak completely enters the third chromatographic column 20, C₃₊ hydrocarbon compositions in the first chromatographic column 5 are blown back by a second carrier gas 9, and then vented by the needle valve 16 through the four-way switching valve 17.

The analysis system further includes a sample storage tank 1, a first pressure relief valve 2. A gas output end of the sample storage tank 1 is connected to the first port of the ten-way switching valve 10 through the first pressure relief valve 2. The transformer oil sample stored in the sample storage tank 1 is subjected to pressure relief by the first pressure relief valve 2, and then flows to the sample loop 3 after passing through the first port of the ten-way switching valve 10 and the tenth port of the ten-way switching valve 10 in sequence.

Further, the analysis system further includes a carrier gas storage device 14, a second pressure relief valve 13, a first planar tee joint 11, and a second planar tee joint 12. The carrier gas storage device 14 is connected to a first interface of the first planar tee joint 11 by the second pressure relief valve 13, a second interface of the first planar tee joint 11 is connected to the seventh port of the ten-way switching valve 10 by a second carrier gas pipeline, a third interface of the first planar tee joint 11 is connected to a first interface of the second planar tee joint 12, a second interface of the second planar tee joint 12 is connected to the fourth port of the ten-way switching valve 10 through a first carrier gas pipeline, and a third interface of the second planar tee joint 12 is connected to a third port of the four-way switching valve 17. A carrier gas stored in the carrier gas storage device 14 is subjected to pressure relief by the second pressure relief valve 13, and then flows to the seventh port of the ten-way switching valve 10 through the first planar tee joint 11, flows to the fourth port of the ten-way switching valve 10 through the first planar tee joint 11 and the second planar tee joint 12 in sequence, or flows to the third port of the four-way switching valve 17 through the first planar tee joint 11 and the second planar tee joint 12 in sequence.

Further, the analysis system further includes a tail gas recovery unit 4. The tail gas recovery unit 4 is connected to a second port of the ten-way switching valve 10.

The first chromatographic column 5 is internally filled with a Porapak R chromatographic support or other equivalent chromatographic support. The second chromatographic column 7 is internally filled with a 5A chromatographic support or other equivalent chromatographic support. The third chromatographic column 20 is internally filled with a Porapak T chromatographic support or other equivalent chromatographic support.

The basic principle of the system for analyzing gas composition content of transformer oil in this embodiment is as follows:

In a sample collection phase: the sample, after flowing out from the sample storage tank 1, is subjected to pressure relief by the first pressure relief valve 2, and then flows to the tail gas recovery unit 4 after passing through the first port of the ten-way switching valve 10, the tenth port of the ten-way switching vale 10, the sample loop 3, the third port of the ten-way switching valve 10 and the second port of the ten-way switching valve 10.

A carrier gas flowing path is as follows: the carrier gas flows out from the carrier gas storage device 14, flows to the first planar tee joint 11 after being subjected to pressure relief by the second pressure relief valve 13, and then flows out from one interface of the first planar tee joint 11 as a second carrier gas 9, and then the second carrier gas 9 flows to the needle valve 16 after passing through the seventh port of the ten-way switching valve 10, the sixth port of the ten-way switching valve 10, the first chromatographic column 5, the ninth port of the ten-way switching valve 10, the eighth port of the ten-way switching valve 10, the first port of the four-way switching valve 17 and the fourth port of the four-way switching valve 17.

A carrier gas flows from the third interface of the first planar tee joint 11 to one interface of the second planar tee joint 12 and flows out as the first carrier gas 6, and the first carrier gas 6 flows to the thermal conductivity cell detector 8 after passing through the fourth port of the ten-way switching valve, the fifth port of the ten-way switching valve 10, and the second chromatographic column 7.

A carrier gas flows out from the third interface of the second planar tee joint 12 as a third carrier gas 18, and the third carrier gas 18 flows to the hydrogen flame ionization detector 15 after passing through the third port of the four-way switching valve 17, the second port of the four-way switching valve 17 and the nickel converter device 19.

According to the present disclosure, the analysis and detection of hydrogen (H₂), oxygen (O₂), nitrogen (N₂), carbon monoxide (CO), methane (CH₄), carbon dioxide (CO₂), acetylene (C₂H₂), ethylene (C₂H₄) and ethane (C₂H₆) in the transformer oil are achieved by a single injection, and C₃₊ hydrocarbon compositions can be vented in time, thus avoiding mutual interference between the compositions.

In this embodiment, the analysis and detection process of the system for analyzing gas composition content of transformer oil in this embodiment is as follows:

An analysis and detection process of hydrogen, oxygen and nitrogen compositions is as follows:

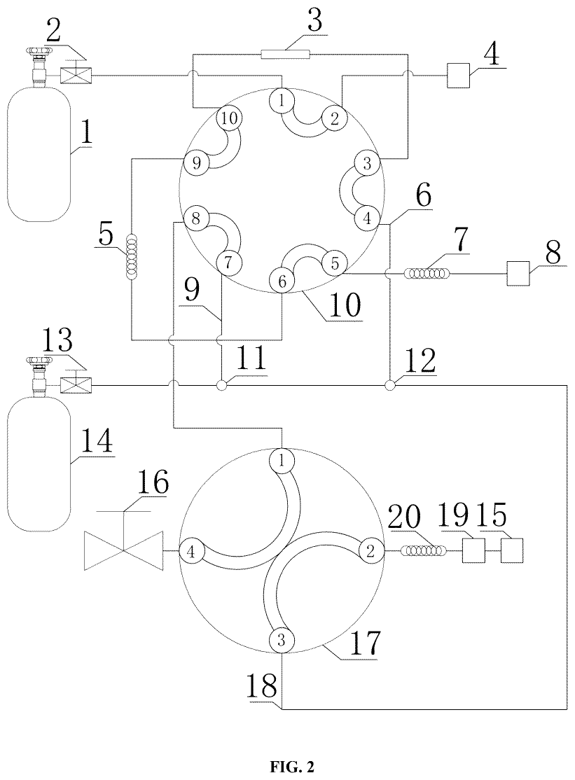

As shown in FIG. 2, the first carrier gas 6 carriers a sample in the sample loop 3 to enter the first chromatographic column 5, the sample is pre-separated into a fitted peak of hydrogen, oxygen and nitrogen, a fitted peak of carbon monoxide, methane, carbon dioxide, and C₂ hydrocarbon compositions, and a fitted peak of C₃₊ hydrocarbon compositions. When the fitted peak of the hydrogen, oxygen and nitrogen completely enters the second chromatographic column 7, the ten-way switching valve 10 is reset to a state shown in FIG. 1, and the hydrogen, oxygen and nitrogen compositions are detected by the thermal conductivity cell detector 8 after being separated by the second chromatographic column 7.

An analysis and detection process of carbon monoxide, methane and carbon dioxide compositions is as follows:

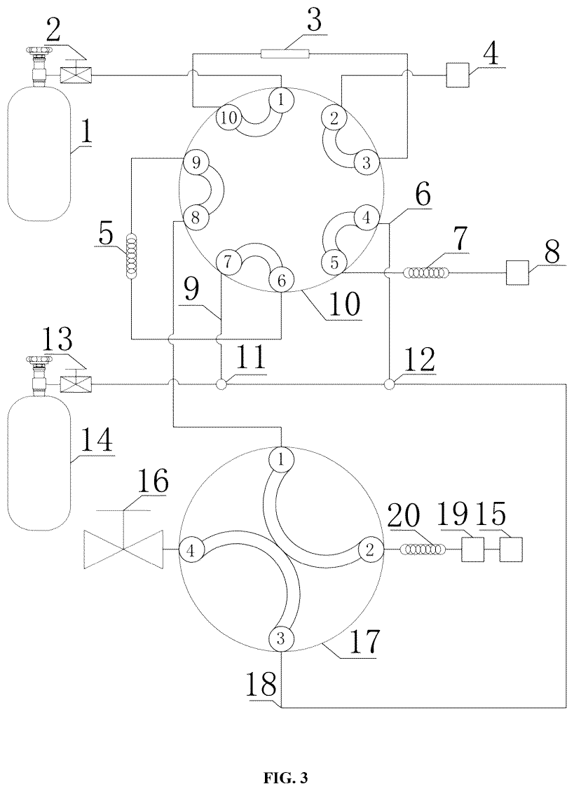

As shown in FIG. 2, the first carrier gas 6 carriers the sample in the sample loop 3 to enter the first chromatographic column 5, and the sample is pre-separated into the fitted peak of hydrogen, oxygen and nitrogen, the fitted peak of carbon monoxide, methane, carbon dioxide, and C₂ hydrocarbon compositions, and the fitted peak of C₃₊ hydrocarbon compositions. When the fitted peak of the hydrogen, oxygen and nitrogen completely enters the second chromatographic column 7, the four-way switching valve 17 is reset to a state shown in FIG. 3. The second carrier gas 9 carriers the fitted peak of carbon monoxide, methane, carbon dioxide and C₂ hydrocarbon compositions in the first chromatographic column 5 to enter the third chromatographic column 20, which is further separated into carbon monoxide, methane, carbon dioxide, ethylene, ethane and acetylene compositions (in which carbon monoxide and carbon dioxide are hydrogenated under the catalysis of a nickel catalyst at a high temperature of 360° C. to generate methane), and detected by the hydrogen flame ionization detector 15.

A venting process of C₃₊ hydrocarbon compositions is as follows:

As shown in FIG. 3, the second carrier gas 9 carries the fitted peak of the carbon monoxide, methane, carbon dioxide, C₂ hydrocarbon compositions in the first chromatographic column 5 to completely enter the third chromatographic column 20, the four-way switching valve 17 is reset to a state in FIG. 1, and the C₃₊ hydrocarbon compositions in the first chromatographic column 5 are blown back by the second carrier gas 9, and then vented by a needle valve 16.

This embodiment of the present disclosure provides a method for analyzing gas composition content of transformer oil, including the following steps:
a sample collection phase:

An interface state of a ten-way switching valve 10 is set to a first interface connection mode, and an interface state of a four-way switching valve 17 is set to a first connection state. The first interface connection mode of the ten-way switching valve 10 is that a first port communicates with a tenth port, a second port communicates with a third port, a fourth port communicates a fifth port, a sixth port communicates with a seventh port, and an eighth port communicates with a ninth port. The first connection state of the four-way switching valve 17 is that a first port communicates with a fourth port, and a second port communicates with a third port.

A transformer oil sample is introduced from the first port of the ten-way switching valve 10, and then flows to a sample loop 3.

A hydrogen, oxygen and nitrogen composition detection phase:

The interface state of the ten-way switching valve 10 is set to a second interface connection mode. The second interface connection mode of the ten-way switching valve 10 is that the first port communicates with the second port, the third port communicates with the fourth port, the fifth port communicates with the sixth port, the seventh port communicates with the eighth port, and the ninth port communicates with the tenth port.

A first carrier gas 6 is introduced from the fourth port of the ten-way switching valve 10, the first carrier gas 6 carries the transformer oil sample in the sample loop 3 to enter a first chromatographic column 5, a first composition fitted peak and a second composition fitted peak are separated in sequence from the transformer oil sample. The first composition fitted peak is a fitted peak of hydrogen, oxygen, and nitrogen, and the second composition fitted peak is a fitted peak of carbon monoxide, methane, carbon dioxide and C₂ hydrocarbon compositions.

After the first composition fitted peak completely enters a second chromatographic column 7, the four-way switching valve 17 is switched to a second connection state, and after hydrogen, oxygen and nitrogen are obtained by separating the first composition fitted peak by the second chromatographic column 7, the contents of hydrogen, oxygen and nitrogen are respectively detected by a thermal conductivity cell detector 8, and the second connection state of the four-way switching valve 17 is that the first port communicates with the second port, and the third port communicates with the fourth port.

A carbon monoxide, methane, carbon dioxide and C₂ hydrocarbon compositions detection phase:

The interface state of the ten-way switching valve 10 is switched to the first interface connection mode.

A second carrier gas 9 is introduced from the seventh port of the ten-way switching valve 10, the second carrier gas 9 carries the second composition fitted peak to completely enter a third chromatographic column 20, and the second composition fitted peak is separated by the third chromatographic column 20 and then enters a carbon composition detection device for determining the contents of carbon monoxide, methane, carbon dioxide and C₂ hydrocarbon.

After the second carrier gas 9 carries the second composition fitted peak to completely enter the third chromatographic column 20, the method further includes the following steps:

The four-way switching valve 17 is switched to the first connection state, C₃₊ hydrocarbon compositions in the first chromatographic column 5 are blown back by the second carrier gas 9 and then vented by a needle valve 16.

The first composition fitted peak and the second composition fitted peak are subsequently separated from the transformer oil sample in the first chromatographic column 5, and then the fitted peak of C₃₊ hydrocarbon components is separated. Finally, the contents of gases are detected in sequence according to a separation order.

Referring to FIG. 2, a gas flow in the hydrogen, oxygen and nitrogen composition detection phase is as follows: the carrier gas storage device 14→the second pressure relief valve 13→the first planar tee joint 11→the second planar tee joint 12→the fourth port of the ten-way switching valve 10→the third port of ten-way switching valve 10→the sample loop 3→the tenth port of the ten-way switching valve 10→the ninth port of the ten-way switching valve 10→the first chromatographic column 5→the sixth port of the ten-way switching valve 10→the fifth port of the ten-way switching valve 10→the second chromatographic column 7→the thermal conductivity cell detector 8.

Referring to FIG. 3, a gas flow in the carbon monoxide, methane, carbon dioxide and C₂ hydrocarbon compositions detection phase is as follows: the carrier gas storage device 14→the second pressure relief valve 13→the first planar tee joint 11→the seventh port of the ten-way switching valve 10→the sixth port of the ten-way switching valve 10→the first chromatographic column 5→the ninth port of the ten-way switching valve 10→the eighth port of the ten-way switching valve 10→the first port of the four-way switching valve 17→the second port of the four-way switching valve 17→the third chromatographic column 20→the nickel converter device 19→the hydrogen flame ionization detector 15.

Referring to FIG. 1, a gas flow in the C₃₊ hydrocarbon compositions detection phase is as follows: the carrier gas storage device 14→the second pressure relief valve 13→the first planar tee joint 11→the seventh port of the ten-way switching valve 10→the sixth port of the ten-way switching valve 10→the first chromatographic column 5→the ninth port of the ten-way switching valve 10→the eighth port of the ten-way switching valve 10→the first port of the four-way switching valve 17→the fourth port of the four-way switching valve 17→the needle valve 16.

Various embodiments in this specification are described in a progressive way, and each embodiment focuses on the differences from other embodiments, so it is only necessary to refer to the same and similar parts of the embodiments. As for the method disclosed in the embodiment, as the method corresponds to the system disclosed in the embodiment, the description is relatively simple, and reference can be made to the description of the method for relevant contents.

Specific examples are used herein for illustration of the principles and embodiments of the present disclosure. The description of the embodiments is merely used to help illustrate the method and its core principles of the present disclosure. In addition, those of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. A system for analyzing gas composition content of transformer oil, comprising:
a ten-way switching valve, a sample loop, a first chromatographic column, a second chromatographic column, a thermal conductivity cell detector, a four-way switching valve, a third chromatographic column, and a carbon composition detection device;
wherein a first port of the ten-way switching valve is a transformer oil sample inlet, a tenth port of the ten-way switching valve is connected to one end of the sample loop, and a third port of the ten-way switching valve is connected to an other end of the sample loop;
a fourth port of the ten-way switching valve is a first carrier gas inlet, a ninth port of the ten-way switching valve is connected to one end of the first chromatographic column, and a sixth port of the ten-way switching valve is connected to an other end of the first chromatographic column;
a fifth port of the ten-way switching valve is connected to one end of the second chromatographic column, and the thermal conductivity cell detector is connected to an other end of the second chromatographic column;
the first chromatographic column is used to separate a dissolved gas in the transformer oil sample to obtain a first composition fitted peak and a second composition fitted peak in sequence;
the first composition fitted peak is a fitted peak of hydrogen, oxygen, and nitrogen;
the second composition fitted peak is a fitted peak of carbon monoxide, methane, carbon dioxide and a C₂ hydrocarbon compositions; the second chromatographic column is used to separate from the first composition fitted peak to obtain hydrogen, oxygen, and nitrogen;
the thermal conductivity cell detector is used to detect contents of hydrogen, oxygen and nitrogen, respectively;
a seventh port of the ten-way switching valve is a second carrier gas inlet, an eighth port of the ten-way switching valve is connected to a first port of the four-way switching valve, a second port of the four-way switching valve is connected to one end of the third chromatographic column, and an other end of the third chromatographic column is connected to the carbon composition detection device;
the third chromatographic column is used to separate from the second composition fitted peak to obtain carbon monoxide, methane, carbon dioxide, ethylene, ethane and acetylene; and
the carbon composition detection device is used to detect contents of carbon monoxide, methane, carbon dioxide, ethylene, ethane and acetylene, respectively.

2. The system for analyzing gas composition content of transformer oil according to claim 1, wherein the carbon composition detection device comprises a nickel converter device, and a hydrogen flame ionization detector;
one end of the nickel converter device is connected to the other end of the third chromatographic column, and an other end of the nickel converter device is connected to the hydrogen flame ionization detector;
the nickel converter device is used to hydrogenate carbon monoxide and carbon dioxide under catalysis of a nickel catalyst to generate methane; and
the hydrogen flame ionization detector is used to detect contents of methane, acetylene, ethylene and ethane, and to detect contents of carbon monoxide and carbon dioxide in the generated methane.

3. The system for analyzing gas composition content of transformer oil according to claim 1, wherein the system further comprises a needle valve;
the needle valve is connected to a fourth port of the four-way switching valve; and
after the second composition fitted peak completely enters the third chromatographic column, C₃₊ hydrocarbon compositions in the first chromatographic column are blown back by a second carrier gas, and then vented by the needle valve through the four-way switching valve.

4. The system for analyzing gas composition content of transformer oil according to claim 1, wherein the system further comprises a sample storage tank, and a first pressure relief valve;
a gas output end of the sample storage tank is connected to the first port of the ten-way switching valve by means of the first pressure relief valve; and
transformer oil sample stored in the sample storage tank is subjected to pressure relief by the first pressure relief valve, and then flows to the sample loop after passing through the first port of the ten-way switching valve and the tenth port of the ten-way switching valve in sequence.

5. The system for analyzing gas composition content of transformer oil according to claim 1, wherein the system further comprises a carrier gas storage device, a second pressure relief valve, a first planar tee joint, and a second planar tee joint;
the carrier gas storage device is connected to a first interface of the first planar tee joint by the second pressure relief valve, a second interface of the first planar tee joint is connected to the seventh port of the ten-way switching valve by a second carrier gas pipeline, a third interface of the first planar tee joint is connected to a first interface of the second planar tee joint, a second interface of the second planar tee joint is connected to the fourth port of the ten-way switching valve through a first carrier gas pipeline, and a third interface of the second planar tee joint is connected to a third port of the four-way switching valve; and
a carrier gas stored in the carrier gas storage device is subjected to pressure relief by the second pressure relief valve, and then flows to the seventh port of the ten-way switching valve through the first planar tee joint, flows to the fourth port of the ten-way switching valve through the first planar tee joint and the second planar tee joint in sequence, or flows to the third port of the four-way switching valve through the first planar tee joint and the second planar tee joint in sequence.

6. The system for analyzing gas composition content of transformer oil according to claim 1, wherein the system further comprises a tail gas recovery unit; and
the tail gas recovery unit is connected to a second port of the ten-way switching valve.

7. The system for analyzing gas composition content of transformer oil according to claim 1, wherein the first chromatographic column is internally filled with a Porapak R chromatographic support;
the second chromatographic column is internally filled with a 5A chromatographic support; and
the third chromatographic column is internally filled with a Porapak T chromatographic support.

8. A method for analyzing gas composition content of transformer oil, comprising:
a sample collection phase:
setting an interface state of a ten-way switching valve to a first interface connection mode, and setting an interface state of a four-way switching valve to a first connection state, wherein the first interface connection mode is that, for the ten-way switching valve, a first port communicates with a tenth port, a second port communicates with a third port, a fourth port communicates a fifth port, a sixth port communicates with a seventh port, and an eighth port communicates with a ninth port; and
the first connection state is that, for the four-way switching valve, a first port communicates with a fourth port, and a second port communicates with a third port;
introducing a transformer oil sample from the first port of the ten-way switching valve, and enabling the transformer oil sample to flow to a sample loop;
a hydrogen, oxygen and nitrogen composition detection phase:
setting the interface state of the ten-way switching valve to a second interface connection mode, wherein the second interface connection mode is that, for the ten-way switching valve, the first port communicates with the second port, the third port communicates with the fourth port, the fifth port communicates with the sixth port, the seventh port communicates with the eighth port, and the ninth port communicates with the tenth port;
introducing a first carrier gas from the fourth port of the ten-way switching valve, carrying, by the first carrier gas, the transformer oil sample in the sample loop to enter a first chromatographic column, and separating a first composition fitted peak and a second composition fitted peak in sequence from the transformer oil sample, wherein the first composition fitted peak is a fitted peak of hydrogen, oxygen and nitrogen, and the second composition fitted peak is a fitted peak of carbon monoxide, methane, carbon dioxide, and a C₂ hydrocarbon compositions;
after the first composition fitted peak completely enters a second chromatographic column, switching the four-way switching valve to a second connection state, and after hydrogen, oxygen and nitrogen are obtained by separating the first composition fitted peak by the second chromatographic column, respectively detecting contents of hydrogen, oxygen and nitrogen by a thermal conductivity cell detector, wherein the second connection state is that, for the four-way switching valve, the first port communicates with the second port, and the third port communicates with the fourth port; and
a carbon monoxide, methane, carbon dioxide and C₂ hydrocarbon composition detection phase:
switching the interface state of the ten-way switching valve to the first interface connection mode; and
introducing a second carrier gas from the seventh port of the ten-way switching valve, carrying, by a second carrier gas, the second composition fitted peak to completely enter a third chromatographic column, wherein the second composition fitted peak is separated by the third chromatographic column and then enters a carbon composition detection device for determining contents of carbon monoxide, methane, carbon dioxide and C₂ hydrocarbon.

9. The method for analyzing gas composition content of transformer oil according to claim 8, wherein after the second carrier gas carries the second composition fitted peak to completely enter the third chromatographic column, the method further comprises:
switching the four-way switching valve to the first connection state, and blowing back, by the second carrier gas, C₃₊ hydrocarbon compositions in the first chromatographic column, thus venting the C₃₊ hydrocarbon compositions by a needle valve.
